SEQUENCE LISTING (20 sequences)

SEQ ID NO: 1 - DNA, 4935 bp, Artificial sequence
  Artificial sequence description plasmid pMK81
ccgacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcacaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatatttctgaaatgagctgttgacaattaatcatcggctcgtataatgtgtggaattgtgagcggataacaatttcacacaggaaacagaattaataatgtatcgattaaataaggaggaataacatatgaggggatcccaccatcaccatcaccacggttctgtcgacgaatccatggacgaattcgagctcggtacccggagatctctcgagctgcagcatgcaagcttcccgggaagaagaagaagaagaagtctgtcgacgaatctctctagtctagactagagcttagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgaaatt

SEQ ID NO: 2 - DNA, 4921 bp, Artificial sequence
  Artificial sequence description plasmid pMK82
ccgacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcacaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatatttctgaaatgagctgttgacaattaatcatcggctcgtataatgtgtggaattgtgagcggataacaatttcacacaggaaacagaattaataatgtatcgattaaataaggaggaataaaccatggacgaattcgagctcggtacccggagatctctcgagctgcagcatgcaagcttcccgggaagaagaagaagaagaagaggcctctcgagatcgaaggtcgggtcgaccaccatcaccatcaccacggatccatctagactagagcttagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgaaatt

SEQ ID NO: 3 - DNA, 4945 bp, Artificial sequence
  Artificial sequence description plasmid pMK83
ccgacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcacaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatatttctgaaatgagctgttgacaattaatcatcggctcgtataatgtgtggaattgtgagcggataacaatttcacacaggaaacagaattaataatgtatcgattaaataaggaggaataacatatgaggggatccaagaagaagaagaagaagggttctgtcgacgaatccatggacgaattcgagctcggtacccggagatctctcgagctgcagcatgcaagcttcccgggatcgagatcgaaggtcgggtcgaccaccatcaccatcaccacggatccatctagactagagcttagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgaaatt

SEQ ID NO: 4 - DNA, 4951 bp, Artificial sequence
  Artificial sequence description plasmid pMK84
ccgacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcacaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatatttctgaaatgagctgttgacaattaatcatcggctcgtataatgtgtggaattgtgagcggataacaatttcacacaggaaacagaattaataatgtatcgattaaataaggaggaataacatatgaggggatcccaccatcaccatcaccacggtggaggtggatctggtggaggtggatctaagaagaagaagaagaagggttctgtcgacgaatccatggacgaattcgagctcggtacccggagatctctcgagctgcagcatgcaagcttcccggggatctagtctagactagagcttagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgaaatt

SEQ ID NO: 5 - DNA, 30 bp, Artificial sequence
  Artificial sequence description spacer arm
aggcctctcgagatcgaaggtcgggtcgac

SEQ ID NO: 6 - DNA, 30 bp, Artificial sequence
  Artificial sequence description spacer arm
ggtggaggtggatctggtggaggtggatct

SEQ ID NO: 7 - DNA, 60 bp, Artificial sequence
  Artificial sequence description spacer arm
aggcctctcgagatcgaaggtcgggtcgacggtggaggtggatctggtggaggtggatct

SEQ ID NO: 8 - DNA, 60 bp, Artificial sequence
  Artificial sequence description spacer arm
ggtggaggtggatctggtggaggtggatctaggcctctcgagatcgaaggtcgggtcgac

SEQ ID NO: 9 - protein, 10 aa, Artificial sequence
  Artificial sequence description sequence encoded by the spacer arm SEQ ID NO5
RPLEIEGRVD

SEQ ID NO: 10 - protein, 10 aa, Artificial sequence
  Artificial sequence description sequence encoded by the spacer arm SEQ ID NO6
GGGGSGGGGS

SEQ ID NO: 11 - protein, 20 aa, Artificial sequence
  Artificial sequence description sequence encoded by the spacer arm SEQ ID NO7
RPLEIEGRVDGGGGSGGGGS

SEQ ID NO: 12 - protein, 20 aa, Artificial sequence
  Artificial sequence description sequence encoded by the spacer arm SEQ ID NO8
GGGGSGGGGSRPLEIEGRVD

SEQ ID NO: 13 - protein, 231 aa, HIV-1 p24 (HXB2 strain)
PIVQNIQGQMVHQAISPRTNLAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVL

SEQ ID NO: 14 - protein, 243 aa, HIV-1 recombinant p24, RH24
MRGSHHHHHHGSVDESMVQNIQGQMVHQAISPRTNLAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGDLV

SEQ ID NO: 15 - protein, 241 aa, HIV-1 recombinant p24, R24H
MVQNIQGQMVHQAISPRTNLAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPPLEIEGRVDHHHHHHGSI

SEQ ID NO: 16 - protein, 252 aa, HIV-1 recombinant p24, RH24K
MRGSHHHHHHGSVDESMVQNIQGQMVHQAISPRTNLAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGKKKKKKSVDESL

SEQ ID NO: 17 - protein, 257 aa, HIV-1 recombinant p24, RK24H
MRGSKKKKKKGSVDESMVQNIQGQMVHQAISPRTNLAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPPLEIEGRVDHHHHHHGSI

SEQ ID NO: 18 - protein, 249 aa, HIV-1 recombinant p24, R24KH
MVQNIQGQMVHQAISPRTNLAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGKKKKKKRPLEIEGRVDHHHHHHGSI

SEQ ID NO: 19 - protein, 264 aa, HIV-1 recombinant p24, R24KsH
MVQNIQGQMVHQAISPRTNLAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGKKKKKKRPLEIEGRVDGGGGSGGGGSGGGGSHHHHHHGSI

SEQ ID NO: 20 - protein, 259 aa, HIV-1 recombinant p24, RHsK24
MRGSHHHHHHGGGGSGGGGSKKKKKKGSVDESMVQNIQGQMVHQAISPRTNLAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGDLV

(19) United States
(12) Patent Application Publication
(10) Pub. No.: US 2005/0033019 A1
(43) Pub. Date: Feb. 10, 2005

(54) Nucleotide sequence coding for a modified protein of interest, expression vector and method for obtaining same

(76) Inventors: Francois Mallet, Villeurbanne (FR); Valerie Cheynet, Verin (FR); Guy Oriol, Saint Chamond (FR); Laure Allard, Le Mans (FR); Armelle Novelli-Rousseau, Seyssins (FR)

Correspondence Address: OLIFF & BERRIDGE, PLC, P.O. BOX 19928, ALEXANDRIA, VA, 22320, US

(21) Appl. No.: 10/495,491

(22) PCT Filed: Nov. 21, 2002
(86) PCT No.: PCT/FR02/04004

(30) Foreign Application Priority Data
Nov. 21, 2001 (FR) 01/15081

(51) Int. Cl. C12Q 1/68; C07H 21/04; C07K 14/705

(52) U.S. Cl. 530/350; 536/23.1; 435/6; 435/320.1; 435/325; 435/69.1

(57) ABSTRACT

The invention concerns a nucleotide sequence coding for a modified protein of interest, said protein of interest having, after purification and immobilization, at least the same biological activity as the native protein of interest and being directly usable, said sequence comprising at least a gene coding for said protein of interest, a nucleotide fragment, called polyK, coding for a succession of at least six lysine residues, and a nucleotide fragment, called polyH, coding for a succession of at least six histidine residues; a vector comprising such a sequence; and a method for obtaining a purifiable and immobilized modified protein of interest.

The invention relates to the determination of a nucleotide sequence encoding a modified protein, to the development of vectors for the expression thereof, and to the uses of the vectors obtained and of the proteins thus expressed.

A modified protein according to the invention is a protein “of interest”, i.e. a protein, or a part of this protein, which it is sought to isolate, for example in diagnostics, or to transport, for example in therapy, in the peptide sequence of which are included, by intercalation and/or addition, at least two series of amino acid residues: a series of at least six lysine residues and a series of at least six histidine residues. In the remainder of the description, the terms “series” and “tag” will be used without differentiating to represent a group of amino acid residues. In the examples which will follow, the protein of interest is the HIV-1 capsid glycoprotein p24, but the subjects of the invention are not of course limited thereto.

According to document WO-A-98/59241, the authors of the present invention have demonstrated that modification of the peptide sequence of the HIV-1 capsid protein p24, by insertion of a tag of six lysine residues, makes it possible to considerably increase the yield from coupling of the protein to the copolymer AMVE67. It has thus been possible to achieve mobilization of 50 molecules of modified protein per copolymer chain.

The immobilization of proteins finds applications in a large number of fields. For example, in chemotherapy, the immobilization of therapeutic proteins makes it possible to increase their lifetime in the blood by limiting proteolytic degradation (Monfardini et al., 1998), but also makes it possible to passively target tumor cells by virtue of the hyperpermeability of these cells (Duncan et al., 1999). In gene therapy, use is made of ligands specific for cell receptors, which are coupled to cationic polymers, in order to transport genes, allowing effective targeting of the cells to be transfected (Varga et al., 2000).

It is known, moreover, that the yield from purification of a protein by immobilized metal ion affinity chromatography (IMAC) is greatly increased when the protein is modified by introducing a tag of at least six histidine residues.

Documents U.S. Pat. No. 5,916,794 and E. Hoculi et al., Bio/Technology, Nature Publishing Co New-York, US, November 1988, pp 1321-1325 describe fusion proteins comprising a protein of interest, namely a restriction endonuclease for U.S. Pat. No. 5,916,794 and dihydrofolate reductase for E. Hoculi et al., and a tag of histidine residues at one or the other of the N- and C-terminal ends of the protein of interest. The presence of this tag makes it possible to increase the yield from isolation of the protein by immobilized metal chelate affinity chromatography.

According to those documents, after isolation, the histidine tag is detached from the protein of interest via the action of thrombin for U.S. Pat. No. 5,916,794, or by chemical or enzymatic cleavage, for example via the action of carboxypeptidase, for E. Hoculi et al., in order to recover, for subsequent use, the protein of interest. This cleavage step is not without risk since, depending on the nature of the amino acids of the protein of interest, and in particular on whether it possesses sites rich in histidine residues, undesired cleavage may occur in the protein. Similarly, the chemical cleavage conditions may be prejudicial to the structure of the protein of interest.

The invention depended on obtaining a modified protein which, at the same time, can be effectively purified by chromatography such as the IMAC technique, can be readily immobilized on a polymer, and has, once purified and immobilized, at least all the biological properties of the native protein for which the modified protein is used and finds a use, without it being necessary to have an additional step using conditions which risk altering the structure of the protein.

Thus, a first subject of the invention is a nucleotide sequence encoding a modified protein of interest, said modified protein of interest having, after purification and immobilization, at least the same biological activity as the native protein of interest and being directly usable, said sequence comprising at least one gene encoding said protein of interest, a “polyK” nucleotide fragment encoding a series of at least six lysine residues, and a “polyH” nucleotide fragment encoding a series of at least six histidine residues.

For the purpose of the present invention, “the same biological activity” is understood as meaning in qualitative terms and in quantitative terms. The applicant has in fact discovered that the insertion and/or the addition both of a histidine tag and of a lysine tag, and then purification and immobilization of the protein thus modified, does not affect the biological function of the protein of interest and alters neither the specificity nor the sensitivity of the protein. This observation is surprising in that, despite the introduction of these two tags representing approximately at least 5% of all the amino acids constituting a protein, for example the HIV capsid protein p24, and despite the immobilization of the protein thus modified, said protein does not appear to lose the conformation which gives it its activity. The term “directly usable” is understood to mean that the modified protein of interest obtained can, after purification and immobilization, be used like the protein of interest, without a prior treatment step to remove one and/or the other of the two histidine and lysine tags.

The invention is of most particular interest in gene therapy, where the protein is coupled to a polymer.

According to the protein under consideration, and in particular depending on the location of its site(s) of activity, in its peptide sequence, the histidine and lysine residue tags, respectively, should be introduced into one and/or the other of the N- and C-terminal ends, or may be intercalated between the epitopes located in said sequence.

Advantageously:
the two tags at least are inserted into, or added to, either the N-terminal end or the C-terminal end of the protein; in this configuration, the two tags may be contiguous or separated by a spacer; or
one of the two tags is inserted into, or added to, the N-terminal end, and the other is inserted into, or added to, the C-terminal end of the protein.

To this effect, a nucleotide sequence of the invention is chosen from the sequences as defined above and also exhibiting the following characteristics:
the nucleotide sequences in which, with respect to said gene encoding the protein of interest, at least one of the two nucleotide fragments, polyK or polyH, is located on the 5′ end of the sequence;
the nucleotide sequences in which, with respect to said gene encoding the protein of interest, the two nucleotide fragments, polyK or polyH, are located on the 5′ end of the sequence; in this configuration, either the polyK nucleotide fragment is located between the polyH nucleotide fragment and the gene, or the polyH nucleotide fragment is located between the polyK nucleotide fragment and the gene;
the nucleotide sequences in which, with respect to said gene encoding the protein of interest, at least one of the two nucleotide fragments, polyK or polyH, is located on the 5′ end of the sequence, and the other of the two nucleotide fragments, polyH or polyK, is located on the 3′ end; in this configuration, either the polyK nucleotide fragment is on the 3′ end and the polyH nucleotide fragment is on the 5′ end, or the polyH nucleotide fragment is on the 3′ end and the polyK nucleotide fragment is on the 5′ end;
the nucleotide sequences in which, with respect to the gene, the two nucleotide fragments, polyK and polyH, are located on the 3′ end of the sequence; in this configuration, either the polyK nucleotide fragment is located between the polyH nucleotide fragment and the gene, or the polyH nucleotide fragment is located between the polyK nucleotide fragment and the gene;
the nucleotide sequences as defined above and in which at least one nucleotide fragment encoding a spacer arm is intercalated between the gene and at least one of the two fragments polyK and polyH, and/or between the two fragments polyK and polyH.

A preferred nucleotide sequence is a sequence in which the polyK fragment encodes a series of six lysine residues, and/or the polyH fragment encodes a series of six histidine residues.

A spacer arm is advantageously chosen from the nucleotide sequences comprising at least any one of SEQ ID NO: 5 to 8. The sequences SEQ ID NO: 9-12 illustrate the peptide sequences encoded by the nucleotide sequences of the spacer arms SEQ ID NO: 5 to 8.

As will be illustrated in the examples, in a particular use for detecting HIV-1, the protein of interest is HIV-1 p24, identified by SEQ ID NO: 13, and the modified protein has a sequence chosen from SEQ ID NO: 14 to 20.

Before disclosing the other subjects of the invention and describing in detail the characteristics and advantages thereof, a definition of certain terms used in the description and the claims is given hereinafter so that the invention and therefore the scope of the protection are clearly delimited.

A “series or tag of amino acid residues” is a short amino acid sequence which is included in the peptide sequence of the native or original protein, at a preferred site, so as to allow this series or tag to be exposed in a relevant manner, while at the same time conserving, or even improving, the biological properties of the native or original protein. In particular according to the invention, the presentation of the histidine residue tag should be favorable with respect to the affinity of this tag for metal ions, as used in the purification technique referred to as IMAC (immobilized metal ion affinity chromatography), and that of the lysine residue tag should be favorable with respect to its attachment to an immobilization phase via a covalent interaction between the tag and reactive functions present on or in said phase.

The expression “intercalation or insertion of a tag” is understood to mean that the tag is introduced within the peptide sequence of the protein of interest, between two amino acids. The expression “addition of a tag” is understood to mean that the tag is “joined onto” the peptide sequence of the protein of interest, at the N- or C-terminal end of said sequence.

In practice, the recombinant modified proteins obtained according to the invention will commonly comprise amino acids which intercalate between the tags, and/or between the tags and the peptide sequence of the native or original protein, without, however, having any effect on the specificity of the tags or on the biological activity of the protein.

The amino acid residues belonging to a tag according to the invention are chosen from natural amino acids and chemically modified amino acids. The chemical modification introduced into the natural amino acid should preserve, or even develop, the specificity of the tag with respect to its role in the attachment. By way of example, mention may be made of replacement of an L amino acid with the corresponding D amino acid, and vice versa; modification of the side chain of the amino acid: in the case of lysine, it may be an acetylation of the amino group of the side chain; modification of the peptide bonds of the tag, such as carba, retro, inverso, retro-inverso, reduced or methyleneoxy bonds.

The immobilization phase to which the attachment of the modified protein is favored by virtue of the lysine residue tag can be a particulate or linear polymer, in particular chosen from homopolymers such as polylysine, polytyrosine; from copolymers such as copolymers of maleic anhydride, copolymers of N-vinylpyrrolidone, natural or synthetic polysaccharides, polynucleotides and copolymers of amino acids such as enzymes. Advantageous polymers are the N-vinylpyrrolidone/N-acryloxysuccinimide copolymer, poly(6-aminoglucose), horseradish peroxidase (HRP) and alkaline phosphatase.

The immobilization phase comprises reactive functions which will interact by covalence with the lysine tag. These reactive functions are chosen from ester, acid, halocarbonyl, sulfhydryl, disulfide, epoxide, halocarbonyl and aldehyde functions.

The immobilization phase can be attached, directly or indirectly, to a solid support by passive adsorption or by covalence.

This solid support can be in any suitable form, such as a plate, a tip, a bead, the bead optionally being radioactive, fluorescent, magnetic and/or conductive, a strip, a glass tube, a well, a sheet, a chip, or the like. The material of the support is preferably chosen from polystyrenes, styrene-butadiene copolymers, styrene-butadiene copolymers mixed with polystyrenes, polypropylenes, polycarbonates, polystyrene-acrylonitrile copolymers and styrene-methyl methacrylate copolymers, from synthetic and natural fibers, and from polysaccharides and cellulose derivatives, glass and silicon, and their derivatives.

A nucleotide sequence according to the invention can be readily synthesized by routine techniques which those skilled in the art know how to implement.

Another subject of the invention is an expression system, such as a vector, for expressing a nucleotide sequence of the invention.

When the protein of interest is HIV-1 capsid p24, a suitable vector has a nucleotide sequence chosen from SEQ ID NO: 1 to 4, preferably the nucleotide sequence is SEQ ID NO: 1 or 3.

The invention also relates to a kit of vectors for the expression of at least two different nucleotide sequences of the invention.

An advantageous kit comprises vectors encoding the expression at least of two nucleotide sequences in which, with respect to said gene encoding the protein of interest, the two nucleotide fragments, polyK or polyH, are located on the 5′ end of the sequence; or of two nucleotide sequences in which, with respect to said gene encoding the protein of interest, at least one of the two nucleotide fragments, polyK or polyH, is located on the 5′ end of the sequence, and the other of the two nucleotide fragments, polyH or polyK, is located on the 3′ end; or else of two nucleotide sequences in which, with respect to the gene, the two nucleotide fragments, polyK and polyH, are located on the 3′ end of the sequence.

Another advantageous kit comprises vectors encoding the expression at least of a nucleotide sequence in which, with respect to said gene encoding the protein of interest, the two nucleotide fragments, polyK or polyH, are located on the 5′ end of the sequence; of a nucleotide sequence in which, with respect to said gene encoding the protein of interest, at least one of the two nucleotide fragments, polyK or polyH, is located on the 5′ end of the sequence, and the other of the two nucleotide fragments, polyH or polyK, is located on the 3′ end; and of a nucleotide sequence in which, with respect to the gene, the two nucleotide fragments, polyK and polyH, are located on the 3′ end of the sequence.

Another subject of the invention is a host cell comprising at least one vector of the invention, in which at least one nucleotide sequence as defined above is expressed.

This ability to obtain and express, in a vector for example, a nucleotide sequence has led the authors to develop a simple method for obtaining a purified and immobilized modified protein of interest, said modified protein of interest having at least the same biological activity as the protein of interest and being directly usable.

This method comprises the following steps:
at least one nucleotide sequence of the invention is provided;
at least the nucleotide sequence is expressed in a suitable expression system;
at least the modified protein thus obtained is purified by metal ion affinity chromatography;
at least the purified modified protein is immobilized.

The authors have also defined a simple and optimal method for obtaining a purified and immobilized modified protein of interest, said modified protein of interest having at least the same biological activity as the protein of interest and being directly usable, said method comprising the following steps:
at least one kit of vectors as defined above, in particular at least one of the advantageous kits, is provided;
the nucleotide sequences are expressed in a suitable expression system;
the modified proteins thus obtained are purified by metal ion affinity chromatography;
the purified modified proteins are immobilized;
the biological activity of the immobilized modified proteins is tested; and
the immobilized modified protein exhibiting the best biological activity is selected.

According to a variant of the method of the invention, said method can also comprise the following steps:
after the purification step, the protein(s) for which the purification yield is highest can be selected, and/or
after the immobilization step, the protein(s) for which the immobilization yield is highest can be selected.

This method makes it possible to select a purified and immobilized modified protein of interest in which the position of the histidine and lysine tags is optimal from the point of view of the biological activity of the modified protein.

A modified protein of interest according to the invention can be readily purified and immobilized and is directly usable, after purification and immobilization, these steps being carried out with very high yields.

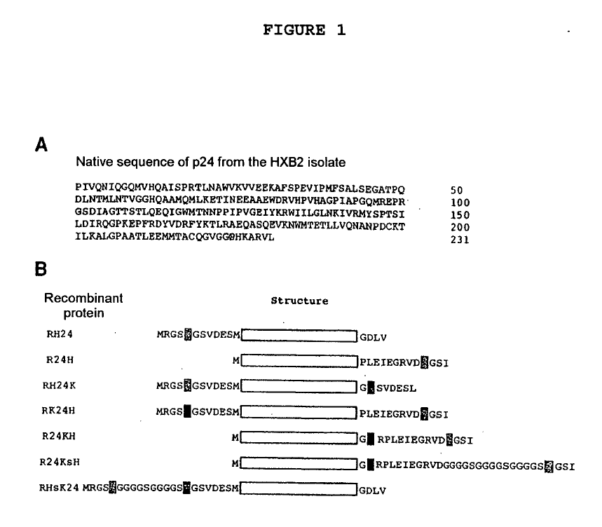

The characteristics and advantages of the various subjects of the invention are illustrated hereinafter, in support of Examples 1 to 6 and of FIGS. 1 to 6, according to which:

FIG. 1 illustrates the native p24 protein and the various modified proteins, as obtained and used according to the present invention.

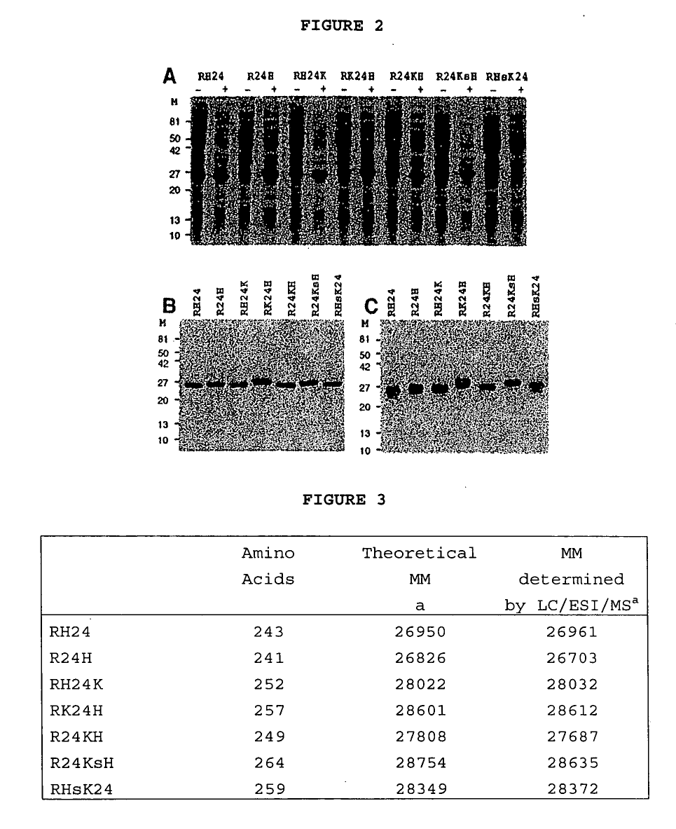

FIG. 2 illustrates the polyacrylamide gel analysis of the expression and of the purification of the recombinant proteins; FIG. 2A shows the level of expression of the various proteins before and after induction with IPTG; FIG. 2B shows the degree of purity of the various proteins after purification by metal chelation for Zn²⁺ ions; FIG. 2C shows the recognition of the purified proteins by a polyclonal antibody after Western blotting transfer onto a nitrocellulose membrane.

FIG. 3 illustrates the physicochemical characteristics of the seven recombinant proteins described in FIG. 1, and more particularly the number of amino acids which make them up and their molecular mass determined by mass spectrometry and compared to the theoretical molecular mass.

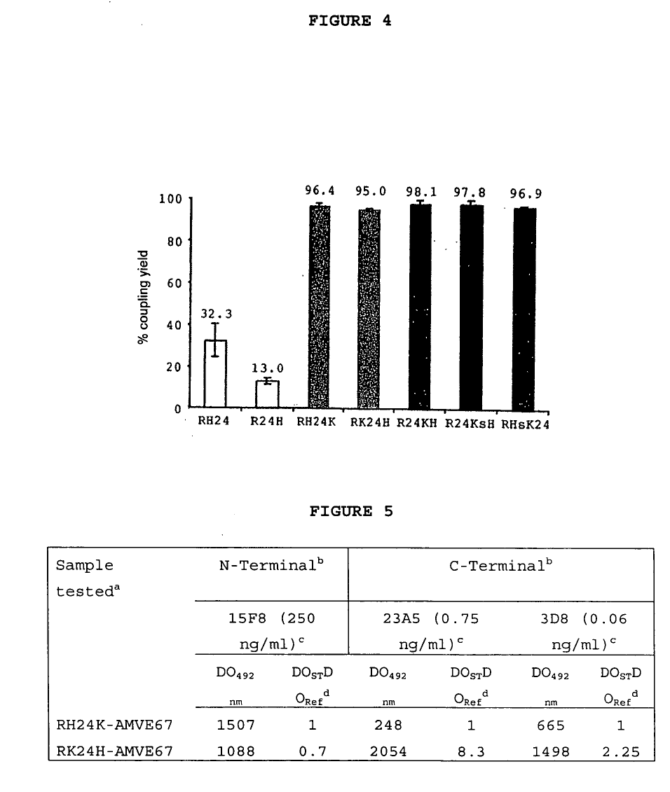

FIG. 4 represents a histogram showing the efficiency of coupling, as a percentage, of the seven recombinant proteins to the AMVE67 polymer.

FIG. 5 illustrates the comparison of the biological reactivities of the conjugates RH24K-AMVE67 and RK24H-AMVE67 in monoclonal antibody capture phase, as a function of the position of the epitope recognized by the antibody.

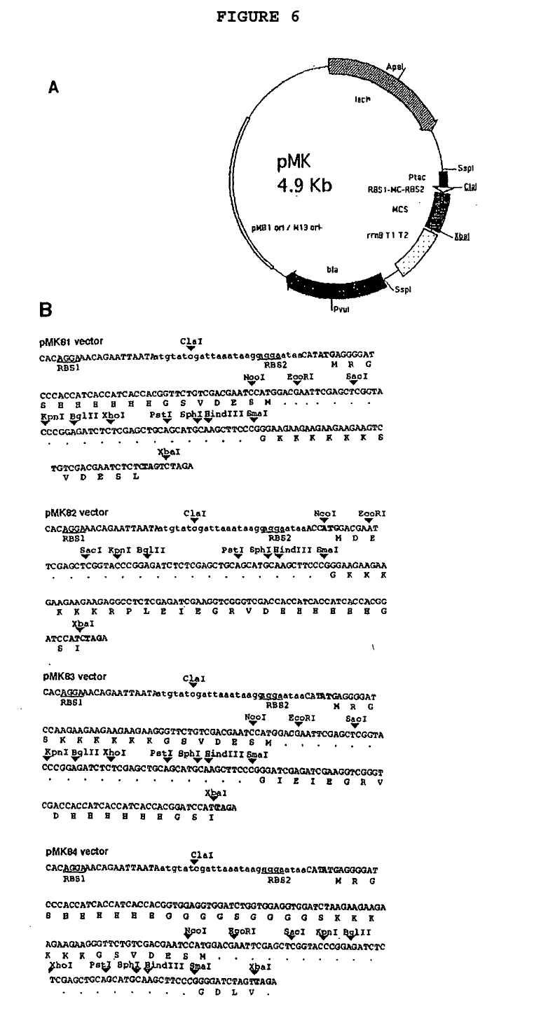

FIG. 6 illustrates the structure of the expression vectors pMK for obtaining modified proteins according to the invention. FIG. 6A shows a diagram of the structure of a vector, and FIG. 6B shows four vector configurations for obtaining the following modified proteins: RH24K, R24 KH, RK24H and RHK24.

### EXAMPLE 1

### Set of Constructs for Obtaining Double-Tagged Proteins

Schematically, the vectors for expressing the tagged recombinant proteins were generated from the expression vector pMR24 obtained by ligation of the NcoI-XbaI fragment of pMH24 (Cheynet et al., 1993) containing the p24 gene, with the NcoI-XbaI fragment of pMR-T7 (WO 98/45449, Arnaud et al., 1997) containing all the sequences regulating replication of the plasmid and the elements for expressing the inserted gene. Suitable oligonucleotide linkers providing the coding information relating to the lysine and/or histidine tags were inserted between ClaI and NcoI in the 5′ position and SmaI and XbaI in the 3′ position, so as to obtain a nucleotide sequence according to the invention. The portion of the p24 gene encoding the polypeptide beginning at amino acid 3 (valine) and terminating at amino acid 224 (proline) is conserved in all the constructs.

The seven inserted nucleotide sequences were designed as follows: all have a nucleotide sequence encoding a series (or tag) of 6 histidine residues, which should allow efficient purification of the protein by metal ion affinity (IMAC for immobilized metal ion affinity chromatography), and five of them have a sequence encoding a series (or tag) of six lysines, in order to allow covalent coupling of the protein to the polymer.

The recombinant modified proteins obtained are as follows:
RH24 encoded by the plasmid pRH24 has a tag of 6 histidine residues at the N-terminal position, illustrated by SEQ ID NO: 14;
R24H encoded by the plasmid pRH24 and pR24H has a tag of 6 histidine residues at the C-terminal position, illustrated by SEQ ID NO: 15;
RH24K encoded by the plasmid pRH24K has a tag of 6 histidine residues at the N-terminal position and a tag of 6 lysine residues at the C-terminal position, illustrated by SEQ ID NO: 16;
RK24H encoded by the plasmid pRK24H has a tag of 6 histidine residues at the C-terminal position and a tag of 6 lysine residues at the N-terminal position, illustrated by SEQ ID NO: 17;
R24 KH encoded by the plasmid pR24 KH has a tag of 6 histidine residues and a tag of 6 lysine residues; both are at the C-terminal position and are contiguous, illustrated by SEQ ID NO: 18;
R24KsH encoded by the plasmid pR24KsH has a tag of 6 lysine residues and a tag of 6 histidine residues; both are at the C-terminal position and are separated by a spacer sequence, illustrated by SEQ ID NO: 19;
RHsK24 encoded by the plasmid pRHsK24 has a tag of 6 histidine residues and a tag of 6 lysine residues; both are at the N-terminal position and are separated by a spacer sequence, illustrated by SEQ ID NO: 20.

The spacer sequence of the recombinant proteins R24KsH and RHsK24 is represented by “s” and consists of a series of four glycine residues and one serine residue, which can be repeated several times.

FIG. 1A describes the peptide sequence of the native p24 protein of the HIV-1 capsid, isolated from the HXB2 strain. The peptide fragment 3-224 represents the sequence conserved in all the recombinant proteins.

FIG. 1B illustrates the structure of the seven recombinant proteins above, the conserved peptide sequence being represented by a white box, the tag of 6 histidine residues being represented by a gray box, and the tag of 6 lysine residues being represented by a black box; the precisely indicated amino acid residues are specific amino acids, outside the previous three boxes and the spacer sequence, which can vary from one recombinant protein to another.

### EXAMPLE 2

### Obtaining of H₆- and K₆-Tagged Recombinant Proteins

E. coli strain XL1 competent bacteria were transformed with the seven plasmids obtained in Example 1, and protein expression was induced by adding isopropyl-β-D-thiogalactopyranoside (IPTG), as previously described (Cheynet et al., 1993, Arnaud et al., 1997). The proteins are extracted, after sonication of the bacterial pellet, in 50 mM Tris buffer, pH 8.0, containing 1 mM EDTA, 10 mM MgCl₂ and 100 mM NaCl, in the presence of antiproteases (10 μg/μl leupeptin and 1.25 μg/μl aprotinin), and then purified by IMAC. The purifications were carried out on a zinc ion-activated Sepharose gel. The recombinant proteins comprising a tag of 6 histidine residues are chelated by the metal ions. The chromatographic system used is an FPLC (Akta Explorer, Pharmacia Biotech). The loading loop is 2 ml. The purifications are carried out by injection of protein diluted ½ in the washing buffer, which is a 67 mM phosphate buffer, pH 7.8, containing 0.5 M NaCl.

The proteins of interest are eluted specifically at approximately pH 4.7 by producing a pH gradient using ammonium acetate buffers, pH 6.0 and pH 3.0. The various purification fractions are collected. 10 μl of each of these fractions are deposited onto Whatman 3MM Chr paper and then stained with Coomassie blue. The fractions (nonretained proteins—purified protein) are then migrated on 12% acrylamide gels after reduction with β-Me and heating for 10 minutes at 95° C., and then stained with Coomassie blue. The fractions containing the highest concentrations of protein of interest are then combined and then dialyzed in a PIERCE Slide-A-Lyzer MWCO 10000 dialysis cassette for 1 hour and then overnight at 4° C., against a 50 mM phosphate buffer, pH 7.8. The protein concentrations are then defined using a calorimetric Bradford Coomassie Plus Assay (PIERCE).

The bacterial protein extracts and the purified proteins are migrated on 12% acrylamide gels after reduction with β-mercaptoethanol and heating for 10 minutes at 95° C., and then stained with Coomassie blue. For the purified proteins, a gel run in parallel is transferred by Western blotting onto a nitrocellulose membrane (Hybond C extra, Amersham Life Science). The nonspecific sites of the membrane are then saturated with Tris buffered saline (TBS)-0.1% Tween, to which 5% of milk has been added. After 3 washes in TBS-T, the membrane is incubated for 2 hours at ambient temperature in the presence of the biotinylated rabbit polyclonal anti-p24 antibody diluted 1/10 000 in TBS-T buffer+5% milk. After 3 washes in TBS-T, the membrane is incubated for 1 hour at ambient temperature in the presence of streptavidin-peroxidase (Jackson ImmunOResearch) at 0.5 g/l diluted 1/3000 in TBS-T buffer+5% milk. Three washes in TBS-T are performed before visualization by ECL+chemiluminescence (Amersham Pharmacia Biotech, RPN2132). Autoradiography for 15 seconds in a dark room is performed on Kodak Biomax MR film.

FIG. 2 illustrates the polyacrylamide gel analysis of the expression and of the purification of the recombinant proteins as follows.

FIG. 2A shows the result of an analysis on 12% acrylamide gel stained with Coomassie blue of the fractions, of the seven recombinant proteins, not induced (−) and induced (+) with 1 mM IPTG for 3 hours at 37° C., with a deposit of 5 μl/well of crude sample. The protein produced is indicated by an arrow (>).

FIG. 2B gives the result of the analysis on 12% acrylamide gel stained with Coomassie blue of the seven recombinant proteins, after purification thereof by Zn²⁺ metal ion chelation, with a deposit of 3 μg/well.

FIG. 2C represents the result of the transfer of the proteins onto a nitrocellulose membrane by Western blotting, after migration on 12% acrylamide gel. The recognition is carried out with a biotinylated rabbit polyclonal antibody diluted 1/10 000 and visualization is carried out by ECL+chemiluminescence, after exposure of the X-ray film for 15 seconds. The deposit was 0.127 μg/well.

The analysis of the expression (FIG. 2A) shows that, for 6 of the 7 expected proteins, the proteins of interest represent approximately 20 to 30% of the total proteins produced by the E. coli bacterium after induction (+), independently of the introduction of the Lys-6 tag (by comparison of RH24K and RH24, and of RK24H and R24H) and of the respective positions of the His-6 and Lys-6 tags (by comparison of RH24K, RK24H, R24 KH and R24KsH). The RHsK24 protein exhibits, for its part, a low level of expression, with less than 5% of the amount of total proteins.

Finally, similar amounts of the recombinant proteins RH24, R24H, RH24K, RK24H, R24 KH and R24KsH are obtained, namely between 2 and 5 mg per gram of biomass for given culturing and extraction conditions, and only 0.4 mg of RHsK24 is obtained, in agreement with its low level of expression. It is observed that, by optimizing the culturing conditions such as the culture volume and the extraction step, yields of 9 to 16 mg per gram of biomass could be obtained for RH24 and RH24K.

The result of the protein purification step is represented in FIG. 2B, and it is observed that the purity on a gel after staining with Coomassie blue is greater than 95%. Recognition on nitrocellulose membrane with a rabbit polyclonal anti-p24 antibody reveals, according to FIG. 2C, that the proteins obtained indeed correspond to those expected. They migrate at a size of approximately 27 kDa, which is in agreement with the expected value. Some proteins exhibit additional weak bands of lower mass and of very weak intensity.

### EXAMPLE 3

### Characterization of the Recombinant Proteins

The purified proteins are then characterized more precisely by mass spectrometry coupled to liquid chromatography (LC/ESI/MS). The analyses were carried out on an API 100 single-quadrupole mass spectrometer, 140B pumps and a 785A detector (Perkin Elmer). The reverse-phase liquid chromatographies were carried out on a C4 column (Vydac Ref 214PT5115, 5 pm particle size). The elution buffers are, for solvent A: 0.1% (v/v) formic acid in water and, for solvent B: formic acid in a water/acetonitrile (5:95 v/v) solution. A gradient of 40 to 60% of B was used.

For each recombinant protein, FIG. 3 gives the number of amino acids, the theoretical (a) molecular masses (MM) determined using the Mac Vector software Version 6.5.3 and the experimental (^{b}) molecular masses determined by mass spectrometry coupled to liquid chromatography (LC/ESI/MS).

The results show that the molecular masses determined by mass spectrometry are in accordance with those expected for the RH24, RH24K, RK24H and RHsK24 proteins, and that, therefore, the proteins used correspond to those deduced from the translation of the modified gene. The R24KsH, R24 KH and R24H proteins exhibit, respectively, a mass deficit of 119, 121 and 123 Da, probably corresponding to the loss of the carboxy-terminal isoleucine. This affects neither of the two tags.

### EXAMPLE 4

### Obtaining of Protein-Polymer Conjugates

The efficiency of coupling of these diversely tagged proteins to copolymers of maleic anhydride was tested. The covalent immobilization of proteins to polymers is carried out by establishing a covalent amide bond between the anhydride groups of the polymer and the primary amines present on the side chains of the lysine residues, as illustrated in the scheme below. However, since the polymer is not water-soluble, it is necessary to dissolve it in anhydrous DMSO (dimethyl sulfoxide) prior to the coupling reaction carried out in 95% aqueous medium. 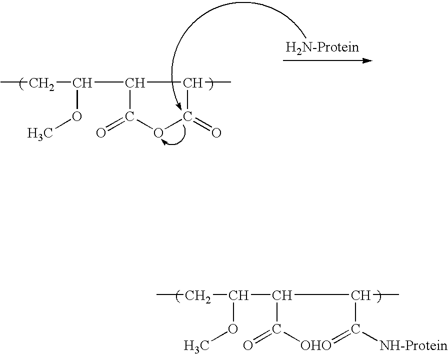
Operating Conditions:
Coupling buffers: 50 mM phosphate, pH 7.8,
Polymer: weigh out 2 mg of AMVE 67 000 copolymer (Polysciences INC batch No. 427393) and dissolve gently in 2 ml of anhydrous DMSO.
Protein: thaw the amount required for the coupling, gently in ice.
   Coupling Protocol:
100 or 36 μg of proteins,
5 μl of polymer at 1 g/l in DMSO (7.46×10⁻¹¹ mol)
qs 105 μl of 50 mM phosphate buffer, pH 7.8.

The covalent coupling reaction is performed spontaneously by incubation for 3 hours at 37° C. on a thermal stirrer.

The conjugates are then characterized as follows.

The samples are filtered in Ultrafree Millex HV 0.45 μm tubes (Millip ore) and then analyzed by steric exclusion chromatography on a Shodex Protein KW 803 column. The chromatographic system is a Kontron HPLC comprising a 422 pump, a 465 automatic injector and a DAD (Diode Array Detector). The elution is performed in 0.1 M phosphate buffer, pH 6.8+0.5% SDS (m/m) with a flow rate of 0.5 ml/min. The detection is carried out by measuring absorbance at 280 (at the concentration used, the polymer does not absorb).

The ratio of the area of the peak corresponding to the protein coupled to the polymer versus the sum of the two peaks corresponding to the cleaved and uncleaved proteins (i.e. the total amount of proteins involved in the reaction) gives the value for the coupling yield (Y).

$$\frac{\begin{matrix}( Area of the protein \text{/} polymer conjugate \\ {peak )}_{280 nm} \times 100\end{matrix}}{\begin{matrix}{( Area of the protein \text{/} polymer conjugate peak )}_{280 nm} + \\ {( Area of the free protein peak )}_{280 nm}\end{matrix}}$$

(Area of the protein/polymer conjugate peak)_{280 nm}×100 (Area of the protein/polymer conjugate peak)_{280 nm}+(Area of the free protein peak)_{280 nm}

The number of proteins per polymer chain is defined by the following relationship: N=n.Y/n′ where n and n′ represent, respectively, the number of moles of proteins and the number of polymer chains in the reaction medium.

The data in FIG. 4 illustrate the yields, as a percentage, from coupling the seven recombinant proteins RH24, R24H, RH24K, RK24H, R24 KH, R24KsH and RHsK24 derived from the HIV-1 capsid protein p24 to the AMVE67 copolymer in 50 mM phosphate buffer, pH 7.8.

The concentrations used are as follows:
[proteins]=0.95 g/l (3.56×10⁻⁹ mol), [AMVE67]=0.048 g/l (7.46×10⁻¹ mol).
□ represents the proteins containing only a tag of 6 histidine residues, ▪ represents the proteins with a tag of 6 histidine residues opposite the tag of 6 lysine residues, ▪ represents the proteins with tags of 6 histidine residues and 6 lysine residues which are contiguous. The experiments were carried out 3 times, the values indicated correspond to the mean plus one standard deviation.

In the absence of lysine residues, the coupling yields are between 10 and 30%. They are greater than 95% when the tag of 6 lysine residues is present on the protein. The presence of a tag of 6 lysine residues therefore makes it possible to considerably improve the coupling efficiency (by comparison of RK24H, R24 KH, R24KsH and RHsK24 with RH24 and R24H), independently of its N- or C-terminal position (comparison of RK24H with RH24K, and RHsK24 with R24KsH), opposite or adjacent to the tag of 6 histidine residues (comparison of RH24K and RK24H with R24 KH, R24KsH and RHsK24).

### EXAMPLE 5

### Bioreactivity of the Proteins Thus Coupled

The improvement in the yield from coupling the Lys-6 proteins to the AMVE67 copolymer suggests that the coupling reaction is region-selective, namely that it involves the lysine residue tag.

The biological reactivity of the conjugates was evaluated as a function of the N- or C-terminal position of the tag and of the N- or C-terminal position of the epitope recognized by the monoclonal antibody. Two proteins were selected for this study, RH24K and RK24H, having, respectively, a tag of six lysine residues at the C-terminal and N-terminal position, and opposite the tag of six histidine residues.

The ELISA protocol was carried out as follows: 100 μl/well of protein-polymer conjugate diluted to 0.25 μg/ml in PBS buffer are immobilized at the bottom of a 96-well microplate (Nunc Immuno^{a} Plate Maxisorp^{a} surface) by overnight incubation at ambient temperature. The nonspecific sites are then saturated for 2 hours at 37° C. with 200 μl/well of a solution of PBS containing 1% (w/v) Régilait™. The wells are then washed 3 times in PBS-0.05% tween. The monoclonal antibodies diluted at the appropriate dilution in PBS buffer-0.05% tween-0.2% Régilait™ are then incubated for 1 hour at 37° C. After 3 washes in PBS-0.05% tween, the peroxidase-labeled anti-mouse conjugate (Jackson ImmunOResearch) diluted 1/2000 in PBS-0.05% tween-1% Régilait™ is incubated for 1 hour at 37° C. Three washes in PBS-0.05% tween are carried out before the visualization during which 100 μl of a solution containing a 30 mg OPD tablet diluted in 10 ml of OPD substrate buffer (Sanofi pasteur) are incubated for 10 min in the dark at ambient temperature. The reaction is then blocked by adding 100 μl/well of 1 N H₂SO₄, and the absorbance values are then read on a spectrophotometer at 492 nm.

The data in FIG. 5 are as follows:

The Table gives the signal obtained by ELISA with a protein-polymer conjugate coating.
^{a}RH24K and RK24H proteins coupled to the AMVE67 polymer.
^{b}Position of the epitope recognized by the monoclonal antibody.
^{c}The detection was carried out using a monoclonal antibody.
^{d}Ratio determined from the OD of the sample tested (OD_{ST}) and from the OD of the reference conjugate RH24K-AMVE67 (OD_{Ref}).

The results show that the ELISA signal is better when the tag is in an opposite position to the epitope recognized by a monoclonal antibody. Thus, the monoclonal antibody which recognizes an epitope located at the N-terminal position (MAb 15F8) exhibits a signal 1.3 times greater for a protein immobilized via its C-terminal region (RH24K) than for a protein immobilized via its N-terminal region (RK24H). Conversely, an antibody which recognizes an epitope located in the C-terminal position exhibits a signal 8.3 times (MAb 23A5) and 2.25 times (MAb 3D8) greater when the protein is immobilized via its N-terminal region (RK24H) than when said protein is immobilized via its C-terminal region (RH24K).

### EXAMPLE 6

### Preparation of a Modified Protein Expression Vector Kit

Given the expression, purification and oriented coupling capacities exhibited by the various double-tagged proteins derived from the p24 model, expression vectors allowing the insertion of a gene of interest for which the three properties would be required were produced. These vectors combine sequences encoding a tag of six histidine residues for efficient purification by metal ion chelation and a tag of six lysine residues for oriented covalent immobilization. According to the use and/or to restrictions imposed by the position of the active site of the protein, the expression vectors proposed exhibit various possible combinations.

The vector pMK81 is derived from the expression vector pH24K by cleavage with NcoI and SmaI, and then by ligation to the sequence of the NcoI-SmaI polyLinker. The vector pMK81 contains, in the 5′ position, a reading frame encoding a His-6 tag, unique cloning sites for the insertion of genes encoding proteins of interest and, in the 3′ position, a reading frame encoding a Lys-6 tag. It is 4935 bp in size.

The vector pMK82 is derived from the expression vector p24 KH by cleavage with NcoI and SmaI, and then by ligation to the NcoI-SmaI polyLinker sequence. The vector pMK82 contains, in the 5′ position, a translation start codon, unique cloning sites for the insertion of genes encoding proteins of interest and, in the 3′ position, a reading frame encoding a Lys-6 His-6 double tag. It is 4921 bp in size.

The vector pMK83 is derived from the expression vector pK24H by cleavage with NcoI and XhoI, and then by ligation to the NcoI-XhoI polyLinker sequence. During construction, the XhoI site was deleted. The double-stranded oligonucleotides were obtained by hybridization of each strand in buffer containing 50 mM NaCl, 6 mM Tris/HCl, pH 7.5, and 8 mM MgCl₂, by heating for 5 minutes at 65° C., and slow cooling at ambient temperature. The vector pMK83 contains, in the 5′ position, a reading frame encoding a Lys-6 tag, unique cloning sites for the insertion of genes encoding proteins of interest and, in the 3′ position, a reading frame encoding a His-6 tag. It is 4945 bp in size.

The vector pMK84 is derived from the expression vector pHK24 by cleavage with NcoI and SmaI, and then by ligation to the sequence of the NcoI-SmaI polyLinker. The vector pMK84 contains, in the 5′ position, a reading frame encoding a His-6 Lys-6 double tag, unique cloning sites for the insertion of genes encoding proteins of interest and, in the 3′ position, a translation stop codon. It is 4951 bp in size.

The characteristics of the vectors represented in FIG. 6 are as follows:

FIG. 6A represents the structure of the pMK expression vectors. Ptac, tac promotor (black box); RBS1-MC-RBS2, minicistron flanked by 2 ribosome-binding sites (RBS) (white arrow); MCS, multiple cloning site (gray box); rrnB T1 T2, strong transcription terminators (dotted box); bla, gene conferring ampicillin resistance (black arrow); pMB1 ori/M13 ori, origins of replication (thin white box); lac q, gene encoding the lacI^{q} repressor (hashed arrow). The ClaI and XbaI restriction sites flanking the MCS are underlined.

FIG. 6B represents the sequences of the expression vectors pMK81, pMK82, pMK83 and pMK84, surrounding the minicistron (RBS1 and RBS2 underlined, the short open reading frame in small characters), the start and stop codons (bold characters) and the restriction sites of the multiple cloning site. The amino acid sequences corresponding to the amino terminal and carboxy terminal regions of the recombinant proteins, including the tags, are indicated.

### BIBLIOGRAPHY

Monfardini C. and F. M. Veronese. 1998 Stabilization of Substances in Circulation (review) Bioconjugate Chem. 9:418-450.
Duncan R. 1999 Polymer conjugates for tumour targeting and intracytoplasmic delivery. The EPR effect as a common gateway? Pharmaceutical Science & Technology Today 2(11): 441-449.
Varga C. M., Wickham T. J., and D. A. Lauffenburger. 2000 Receptor-mediated targeting of gene delivery vectors: Insights from molecular mechanisms for improved vehicle design (Review). Biotechnology and Bioengineering 70(6): 593-605
Ladaviere C., T. Delair, A. Domard, A. Novelli-Rousseau, B. Mandrand and F. Mallet. 1998. Covalent immobilization of proteins onto (maleic anhydride-alt-methyl vinyl ether) copolymers: enhanced immobilization of recombinant proteins. Bioconjug Chem 9(6):655-661.
Laure Allard, Valérie Cheynet, Guy Oriol, Laurent Véron, Francoise Merlier, Gérald Scrémin, Bernard Mandrand, Thierry Delair and Franois Mallet 2001 Mechanisms Leading to an Oriented Immobilization of Recombinant Proteins Derived from the p24 Capsid of HIV-1 onto Copolymers. Bioconjug Chem in press
Cheynet, V., B. Verrier, and F. Mallet, 1993. Overexpression of HIV-1 proteins in Escherichia coli by a modified expression vector and their one-step purification. Prot Express Purif 4:367-372.
Berthet-Colominas C., S. Monaco, A. Novelli, G. Sibai, F. Mallet and S. Cusack. 1999. Head-to-tail dimers and interdomain flexibility revealed by the crystal structure of HIV-1 capsid protein (p24) complexed with a monoclonal antibody Fab. EMBO 18(5): 1124-1136.
Arnaud N., V. Cheynet, G. Oriol, B. Mandrand and F. Mallet. 1997. Construction and expression of a modular gene encoding bacteriophage T7 RNA polymerase. Gene 199(1-2):149-156.
Ganachaud F., Mouterde G, Delair T, Elaissari A. and Pichot C. 1995 Preparation and characterization of cationic polystyrene latex particles of different aminated surface charges. Polymers for Advanced Technologies 6: 480-488.

1. A method for obtaining a purified and immobilized modified protein of interest, said protein of interest having, after purification and immobilization, at least the same biological activity as the native protein of interest and being directly usable, said method being characterized in that it comprises the following steps:
at least two nucleotide sequences encoding said modified protein of interest, comprising at least one gene encoding said protein of interest, a “polyK” nucleotide fragment encoding a series of at least six lysine residues and a “polyH” nucleotide fragment encoding a series of at least six histidine residues, are provided, the two sequences, chosen from different groups, being chosen from:
(a) the nucleotide sequences in which, with respect to the gene, the two nucleotide fragments, polyK or polyH, are located on the 5′ end of the sequence;
(b) the sequences in which, with respect to the gene, one of the two nucleotide fragments, polyK or polyH, is located on the 5′ end of the sequence, and the other is located on the 3′ end;
(c) the sequences in which, with respect to the gene, the two nucleotide fragments, polyK and polyH, are located on the 3′ end of the sequence;
the nucleotide sequences are expressed in a suitable expression system;
the modified proteins thus obtained are purified by metal ion affinity chromatography;
the purified modified proteins are immobilized on a linear or particulate polymer;
the biological activity of the immobilized modified proteins is tested; and
the immobilized modified protein exhibiting the best biological activity is selected.

2. The method as claimed in claim 1, characterized in that it also comprises at least one of the following steps:
after the purification step, the protein(s) for which the purification yield is highest is (are) selected, and/or
after the immobilization step, the protein(s) for which the immobilization yield is highest is (are) selected.

3. The method as claimed in claim 1, characterized in that, according to (a), the polyK nucleotide fragment is located between the polyH nucleotide fragment and the gene.

4. The method as claimed in claim 1, characterized in that, according to (a), the polyH nucleotide fragment is located between the polyK nucleotide fragment and the gene.

5. The method as claimed in claim 1, characterized in that, according to (b), the polyK nucleotide fragment is located on the 5′ end and the polyH nucleotide fragment is located on the 3′ end.

6. The method as claimed in claim 1, characterized in that, according to (b), the polyH nucleotide fragment is located on the 5′ end and the polyK nucleotide fragment is located on the 3′ end.

7. The method as claimed in claim 1, characterized in that, according to (c), the polyK nucleotide fragment is located between the polyH nucleotide fragment and the gene.

8. The method as claimed in claim 1, characterized in that, according to (c), the polyH nucleotide fragment is located between the polyK nucleotide fragment and the gene.

9. The method as claimed in claim 1, characterized in that, according to (a) or (c), the series of at least six lysine residues and the series of at least six histidine residues are contiguous.

10. The method as claimed in claim 1, characterized in that the polyK fragment encodes a series of six lysine residues, and/or the polyH fragment encodes a series of six histidine residues.

11. The method as claimed in claim 1, characterized in that at least one nucleotide fragment encoding a spacer arm is intercalated between the gene and at least one of the two fragments polyK and polyH and/or between the two fragments polyK and polyH.

12. The method as claimed in claim 11, characterized in that the spacer arm is chosen from the nucleotide sequences comprising at least any one of SEQ ID NO: 5 to 8.

13. The method as claimed in claim 1, characterized in that the protein of interest is the HIV-1 p24 glycoprotein, identified by SEQ ID NO: 13.

14. The method as claimed in claim 13, characterized in that the modified protein has a sequence chosen from SEQ ID NO: 15 to 20.

15. A kit of at least two vectors for the expression of at least two different nucleotide sequences chosen from different groups from the groups (a), (b) and (c) as defined in claim 1.

16. The kit as claimed in claim 15, characterized in that the vectors have a nucleotide sequence chosen from SEQ ID NO: 1 to 4.

17-19. (Cancelled)
